(19) United States
(12) Patent Application Publication
(10) Pub. No.: US 2023/0093239 A1
(43) Pub. Date: Mar. 23, 2023

(54) TARGET ANALYSIS KIT AND ANALYSIS METHOD USING SAME

(71) Applicant: NEC Corporation, Minato-ku, Tokyo (JP)

(72) Inventor: Kenji MIYAZAKI, Tokyo (JP)

(73) Assignee: NEC Corporation, Minato-ku, Tokyo (JP)

(21) Appl. No.: 17/802,264

(22) PCT Filed: Feb. 3, 2021
(86) PCT No.: PCT/JP2021/003906

§ 371 Date: Aug. 25, 2022

(30) Foreign Application Priority Data
Mar. 26, 2020 (JP) 2020-056896

(51) Int. Cl. G01N 33/551; G01N 19/00; G01L 1/18

(52) U.S. Cl. CPC G01N 33/551; G01N 19/00; G01L 1/18

(57) ABSTRACT

The present invention provides an analysis kit including a membrane-type surface stress sensor that can obtain a strong electrical signal as compared with a membrane-type surface stress sensor having a binding substance capable of binding to a target immobilized thereon. A target analysis kit of the present invention includes: a first binding substance that binds to a target; and a membrane-type surface stress sensor, wherein the membrane-type surface stress sensor includes: a second binding substance; a membrane; and a sensor substrate, wherein the second binding substance is a substance that binds to a target and is immobilized to the membrane, the membrane is a membrane that deforms upon binding of the target to the second binding substance, the sensor substrate has a support region, the support region supports the membrane and has a piezoresistive element, and the piezoresistive element is an element for detecting deformation of the membrane.

### TECHNICAL FIELD

The present invention relates to a target analysis kit and an analysis method using the same.

### BACKGROUND ART

In a wide variety of fields such as food, medicine, and the like, detection of targets is important, and various methods have been proposed. In recent years, a membrane-type surface stress sensor has attracted attention (see Patent Literature 1). The membrane-type surface stress sensor can analyze the presence or absence or the amount of a target by, for example, binding a target to a membrane such as a silicon membrane, deforming the membrane, and measuring a variation in electric resistance due to the deformation. However, there is a demand for further improvement of the method for binding a target to the membrane from the viewpoint of, for example, improvement of analysis accuracy and expansion of target to be applied.

### Citation List

### Patent Literature

Patent Literature 1: WO2011/148774

### SUMMARY OF INVENTION

### Technical Problem

Hence, the inventors of the present invention invented a membrane-type surface stress sensor (hereinafter referred to as “MSS”) having a new form for binding a target, specifically, a MSS having an aptamer capable of binding to a target immobilized thereon. In the MSS, when a target is present in a sample liquid being in contact with, the target binds to the aptamer to form a complex, so that the stress of the MSS to the membrane becomes relatively larger than the MSS unbound to the target and the distortion of the membrane becomes large. Thus, in the MSS, by applying a voltage to the MSS to measure the resistance value, the binding of the target can be measured as a change in a resistance value, i.e., an electrical signal. Therefore, according to the MSS, the target in the sample liquid can be analyzed.

However, when analyzing various targets using the MSS, enhancement of the electrical signal is considered necessary. With the foregoing in mind, it is an object of the present invention to provide an analysis kit including a MSS that can obtain a strong electrical signal as compared with a MSS having a binding substance capable of binding to a target immobilized thereon.

### Solution to Problem

In order to achieve the above object, the present invention provides a target analysis kit (hereinafter, referred to as an “analysis kit”), including:

a first binding substance that binds to a target; and

a membrane-type surface stress sensor, wherein

the membrane-type surface stress sensor includes:
a second binding substance;
a membrane; and
a sensor substrate, wherein
the second binding substance is a substance that binds to a target and is immobilized to the membrane,
the membrane is a membrane that deforms upon binding of the target to the second binding substance,
the sensor substrate has a support region,
the support region supports the membrane and has a piezoresistive element, and
the piezoresistive element is an element for detecting deformation of the membrane.

The present invention also provides a method for analyzing a target (hereinafter, referred to as an “analysis method”), including the steps of:

forming a complex of a target in the sample liquid, the first binding substance, and the second binding substance by bringing a sample liquid into contact with the analysis kit according to the present invention;

applying a voltage to the membrane-type surface stress sensor in a liquid phase; and

analyzing the target in the sample liquid by measuring a stress change of the piezoresistive element in the membrane-type surface stress sensor.

### Advantageous Effects of Invention

According to the present invention, a strong electrical signal can be obtained as compared with a MSS having a binding substance capable of binding to a target immobilized thereon.

### BRIEF DESCRIPTION OF DRAWINGS

FIG. 1 is a diagram showing a mechanism by which a MSS signal enhances in the present invention.

FIG. 2 is a schematic diagram showing a general configuration of the MSS.

FIG. 3 is a schematic diagram showing the structure of the MSS in Reference Example 1.

FIG. 4 shows graphs showing the voltage of the MSS in Reference Example 1.

FIG. 5 shows a graph showing the voltage of the MSS in Example 1.

### DESCRIPTION OF EMBODIMENTS

In the present invention, hereinafter, a “membrane-type surface stress sensor” is also referred to as a MSS. In a so-called MSS, a membrane having a binding property to a target is supported by a support having a piezoresistive element. When the target binds to the membrane, the membrane suffers stress due to the binding, and the membrane deforms due to the generation of distortion or the like (generation of distortion). A stress is generated in the piezoresistive element of the support that supports the membrane depending on the amount of deformation of the membrane, and the resistance value of the piezoresistive element changes in proportion to the stress. Therefore, by applying a voltage to the MSS and measuring an electrical signal accompanying a change in resistance value, the MSS sensor can indirectly and qualitatively analyze the presence or absence of the target bonded to the membrane. In addition, by applying a voltage to the MSS and measuring an electrical signal accompanying a change in resistance value, the MSS sensor can quantitatively analyze the amount of the target bonded to the membrane. The present invention is characterized in that a second binding substance that binds to a target is used in such a MSS, specifically, the second binding substance is immobilized to the membrane to bind the target to the MSS via the second binding substance. Therefore, the configuration of the MSS of the present invention is not particularly limited except for immobilizing the second binding substance to the membrane, and existing configurations and configurations in future that exhibit similar functions can be utilized.

In the present invention, the “target” is not particularly limited and can be determined freely. The target may be, for example, a substance that can be in contact with the first binding substance and the second binding substance in a liquid, i.e., in a liquid phase. The target has, for example, one or more regions to which the binding substances bind, i.e., one or more epitopes of the binding substances. When the epitope of the first binding substance is different from the epitope of the second binding substance, the target has one or more epitopes of the first binding substances and one or more epitopes of the second binding substances, for example. On the other hand, if the epitope of the first binding substance is the same or partially overlapping with the epitope of the second binding substance, the target has a plurality of epitopes of the first binding substances and the second binding substances. The partial overlapping means, for example, a state in which a portion of a recognition site of the first binding substance and a portion of a recognition site of the second binding substance overlap with each other in the target.

In the target, it is desirable that the epitope of the first binding substance and the epitope of the second binding substance are set in such a manner that competition does not occur in binding of the first binding substance and the second binding substance to the target. The competition means, for example, that binding of the first binding substance to a target partially or completely inhibits binding of the second binding substance to the target, and/or binding of the second binding substance to a target partially or completely inhibits binding of the first binding substance to the target. The competition can be determined, when labeling the first binding substance and the second binding substance and reacting the target immobilized to a plate in the presence of the first binding substance and the second binding substance, based on whether the detection of the label significantly decreases as compared to reacting the target immobilized to a plate in the presence of the first binding substance or the second binding substance alone, for example. If the detection of the label significantly decreases, it can be determined that the first binding substance and the second binding substance can be determined to be competing. On the other hand, if the detection of the label does not significantly decrease, i.e., there is no significant difference, it can be determined that the first binding substance and the second binding substance can be determined to be not competing.

In the present invention, examples of the target include microorganisms including bacteria such as anthrax, Escherichia coli, Salmonella, Escherichia coli, and the like; viruses such as influenza virus, and the like; and allergens. Examples of the allergen include grains such as wheat, and the like; eggs; meat; fish; shellfish; vegetables; fruits; milk; beans such as peanuts, and the like; and pollens such as cedar pollen, cypress pollen, and the like. The type of the target is not particularly limited, and examples thereof include polymer compounds such as a protein, a sugar chain, a nucleic acid, a polymer, and the like; and low-molecular compounds. When the target is a microorganism, a virus, or an allergen, the target generally has a plurality of identical structures such as identical proteins, lipids, nucleic acids, and the like, so that it can be said that the target has, for example, a plurality of epitopes of the first binding substances and/or the second binding substances. On the other hand, when the target is a monomer of a protein, a sugar chain, or a nucleic acid, the target has one identical structure, so that it can be said that the target has, for example, one epitope of the first binding substance and/or the second binding substance because the target has one identical structure.

In the present invention, the “binding substance” may be any substance capable of binding to a target, that is, a binding substance. Examples of the binding substance include antibodies and aptamers or the like. When the target is a receptor or a ligand thereof, the binding substance may be a ligand or a receptor, respectively. When a receptor for ligand is used as the binding substance, the receptor may be a fusion protein with a Fc region of an immunoglobulin, i.e., a receptor-Fc fusion protein, and preferably a fusion protein with a Fc region of an IgG protein, i.e., a receptor-IgG Fc. The Fc fusion protein can be prepared, for example, by linking an amino acid at the C-terminal of the acceptor directly or via a linker with an amino acid at the N-terminal in a CL region or a CH1 region of an immunoglobulin.

In the present invention, the “antibody” may also be referred to as a soluble form of immunoglobulin having a binding property to a target. Examples of the type of the antibody include IgA, IgD, IgE, IgG, and IgM. Examples of IgA include IgA1 and IgA2. Examples of IgG include IgG1, IgG2, IgG3, and IgG4. The antibody may be an antigen-binding fragment thereof, i.e., a partial peptide of an antibody having a binding property to the target. The antigen-binding fragment is, for example, a portion of the antibody, more specifically a polypeptide having a binding region or variable region of the antibody. Examples of the antigen-binding fragment include Fab, Fab′, F(ab′)₂, Fv fragment, rIgG (semi-IgG) fragment, single-chain antibody (scFv), double variable domain antibody (DVD-Ig™), diabody, triabody, tetrabody, tandab, flexibody that is a combination of scFv and diabody, tandem scFv (e.g., BiTE®, manufactured by Micromet), DART® (manufactured by MacroGenics), Fcab™ or mAb² (manufactured by F-star), Fc engineering antibody (manufactured by Xencor), and DuoBody® (manufactured by Genmab). As the antibody, a known antibody having a binding property to a target or an antigen-binding fragment thereof may be used, or a new antibody or an antigen-binding fragment thereof obtained by immunizing an animal or the like with a target may be used. Further, the antibody may be a monoclonal antibody or a polyclonal antibody. The antibody may be a fraction from blood, such as serum, plasma, or the like containing an antibody capable of binding to a target.

In the present invention, an “aptamer” is a nucleic acid molecule having a binding property to a target. The aptamer can also be, for example, a nucleic acid molecule that specifically binds to a target. Examples of the constituent unit of the aptamer include nucleotide residues and non-nucleotide residues. Examples of the nucleotide residue include deoxyribonucleotide residues and ribonucleotide residues, wherein the nucleotide residues may be modified or unmodified, for example. Examples of the aptamer include DNA aptamers consisting of deoxyribonucleotide residues, RNA aptamers consisting of ribonucleotide residues, aptamers including both deoxyribonucleotide residues and ribonucleotide residues, and aptamers including modified nucleotide residues. The length of the aptamer is not particularly limited and is, for example, from 10 to 200 bases. For example, existing aptamers may be used as the aptamer to the target, or aptamers newly obtained by a SELEX method or the like may be used depending on the target, for example.

In the present invention, “binding to” or “capable of binding to” may mean that a binding substance of interest is actually bonded to a binding object (target) to be bound to the binding substance or a binding substance of interest is bonded to a binding object (target) to be bound to the binding substance in a simulation using a molecular docking method or the like, and the former is preferable. Binding of the binding substance to the binding object can be detected using, for example, a method of analyzing an interaction between proteins, and can be detected using, for example, a method utilizing an antibody-antigen reaction such as co-immunoprecipitation, pull-down assay, ELISA method, flow cytometry, or the like. As a specific example, binding of the binding substance to the binding object can be detected, for example, by bringing a cell expressing the binding object into contact with a labeled binding substance and then detecting a label in the cell.

The first binding substance is preferably an aptamer or antibody. Also, the second binding substance is preferably an aptamer or antibody.

The epitopes of the first binding substance and the second binding substance can be appropriately set based on the type of the target and its structure. The epitopes of the first binding substance and the second binding substance may be the same or different. If the target has a plurality of structures common within the target, the epitopes of the first binding substance and the second binding substance can be set to the same epitopes, for example. On the other hand, if the target does not have a plurality of structures common within the target, the epitopes of the first binding substance and the second binding substance can be set to different epitopes, for example

In the analysis kit of the present invention, the first binding substance may be contained separately from the MSS or may be disposed on a membrane of the MSS, for example.

The first binding substance may be labeled. The label is not particularly limited, and examples thereof include a fluorescent substance, a dye, an isotope, and an enzyme. Examples of the fluorescent substance include chromophores such as pyrene, TAMRA, fluorescein, Cy3 dye, Cy5 dye, FAM dye, rhodamine dye, Texas red dye, JOE, MAX, HEX, TYE, and the like, and examples of the dye include Alexa dyes such as Alexa488, Alexa647, and the like. Examples of the enzyme include luciferase, alkaline phosphatase, peroxidase, β-galactosidase, and glucuronidase. Since the first binding substance increases in weight by being labeled, it is possible to increase the weight of the complex of the second binding substance and the target at the time of forming the complex as compared with the case of using an unlabeled first binding substance. As the weight increases, the stress on the membrane of the MSS relatively increases and the distortion increases. As a result, when the labeled first binding substance is used, the signal detected when a voltage is applied to the MSS can be further enhanced.

Preferably, the first binding substance is immobilized to a carrier. Examples of the carrier include beads and particles. The material of the carrier is not particularly limited, and examples thereof include metal and plastic. Specific examples of the carrier include beads or particles made of polystyrene, beads or particles made of silica, beads or particles made of agarose, beads or particles made of glass, beads or particles made of acrylic resin, beads or particles made of polyvinyl alcohol resin, and beads or particles made of polycarbonate. The carrier may be a magnetic bead.

The size of the label and carrier may be such that, for example, the labeled first binding substance and the first binding substance immobilized to the carrier are diffusible in a liquid phase. As a specific example, the size of the label and carrier is, for example, 1 nm to 100 μm or 10 nm to 100 μm.

It is preferable that the weight of the label and carrier be heavier than the target, for example. Thus, the present invention can further enhance a signal detected when a voltage is applied to the MSS.

When the first binding substance is a nucleic acid, the label and carrier are bonded to at least one of the 5′ and 3′ ends of the nucleic acid, for example. On the other hand, when the first binding substance is a protein, the label and carrier are bonded to the N-terminal, C-terminal, or side chain of the protein, for example.

The label and carrier are bonded to the first binding substance directly or indirectly, for example. In the case of the indirect binding, the label and carrier are bonded via a linker.

The analysis kit of the present invention may further include buffers, instructions, and the like.

In the analysis kit of the present invention, the first binding substance and the MSS may be contained separately or may be contained together. In the latter case, the first binding substance may be disposed, for example, on a MSS membrane of the MSS.

In the present invention, the “sample liquid” is not particularly limited as long as it is a liquid. When a collected sample is a liquid, the collected sample may be used as a liquid sample as it is or after diluting, suspending, dispersing, or the like with a liquid solvent. When a collected sample is a solid, for example, a liquid sample may be prepared by dissolving, suspending, dispersing, or the like with a liquid solvent. In addition, when a collected sample is a gas, for example, a liquid sample may be prepared by concentrating an aerosol in the gas or may be prepared by further dissolving, suspending, dispersing, or the like with a liquid solvent. The type of the liquid solvent is not particularly limited, and is, for example, a solvent which hardly affects the binding between the first binding substance and the second binding substance and a target, and specific examples of the liquid solvent include water and a buffer. Examples of the collected sample include food, blood, urine, saliva, body fluid, soil, drainage, tap water, pond, river, and air. The sample liquid may be, for example, a liquid containing a target, a liquid containing no target, or a liquid of unknown whether it contains a target.

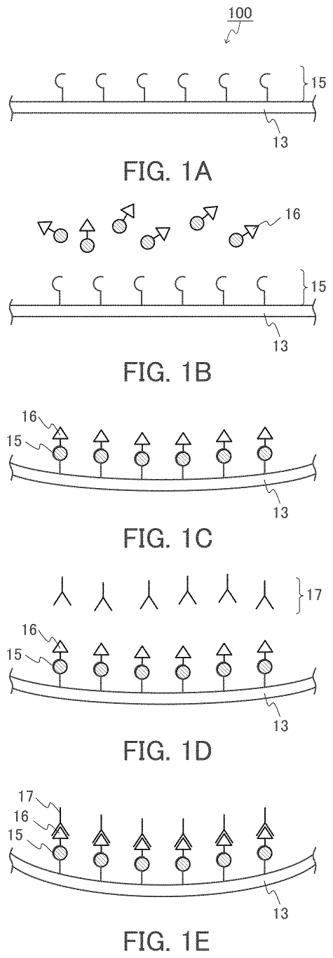

Next, an estimated mechanism, by which a strong electrical signal can be obtained by an analysis and analysis method (hereinafter, referred to as “analysis of the present invention”) using an analysis kit of the present invention as compared with an analysis (hereinafter, referred to as “analysis of the reference example”) using only a MSS having a binding substance (second binding substance) capable of binding to a target immobilized thereon, will be described with reference to FIG. 1. However, the present invention is not limited in any way to the following estimated mechanisms. In addition, while a case of using an aptamer as the second binding substance will be described as an example, it is estimated that the analysis of the present invention can obtain a strong electrical signal as compared with the analysis using only a MSS having a binding substance capable of binding to a target immobilized thereon by the same mechanism as described above even when other binding substances such as an antibody and the like are used as the second binding substance.

First, common parts in the analyses of the present invention and the reference example will be described. In the analyses of the present invention and the reference example, as shown in (A) of FIG. 1, a MSS 100 including a membrane 13 of MSS (hereinafter, referred to as “MSS membrane”) having aptamers 15 (second binding substances) immobilized thereon is used. Next, a sample liquid containing targets 16 is dropped on the MSS membrane 13 as shown in (B) of FIG. 1. Then, since the aptamer 15 is capable of binding to the target 16, a first complex as shown in (C) of FIG. 1 is formed. Also, as the first complex is formed, the weight of the target 16 is loaded onto the MSS membrane 13 via the aptamer 15. Thus, as shown in (C) of FIG. 1, stress is applied to the MSS membrane 13, and the distortion is generated. In the analysis of the reference example, a signal caused by distortion generated when shifting from (B) of FIG. 1 to (C) of FIG. 1 is detected.

The analysis of the present invention further drops aptamers 17 (first binding substances) to the MSS membrane 13 of (C) of FIG. 1, as shown in (D) of FIG. 1. Then, since the aptamer 17 is capable of binding to the first complex of the aptamer 15 and the target 16, a complex of the aptamer 15, the target 16, and the aptamer 17 is formed as shown in (E) of FIG. 1. Also, as the complex is formed, the weight of aptamer 17 is loaded onto the MSS membrane 13 via the first complex of the aptamer 15 and target 16.Thus, as shown in (E) of FIG. 1, further stress is applied to the MSS membrane13,resulting in further distortion. Then, in the analysis of the present invention, a signal caused by a distortion generated when shifting from (B) of FIG. 1 to (E) of FIG. 1 is detected.

As shown in FIG. 1, the analysis of the present invention will detect greater distortion of the MSS membrane13compared to the analysis of the reference example. Therefore, it is estimated that the analysis of the present invention can further enhance the signal detected when a voltage is applied to the MSS100compared to the analysis of the reference example.

While an example in which a voltage is applied to the MSS 100 from the state of (B) of FIG. 1 to detect a signal is described with reference to FIG. 1, the present invention is not limited thereto, and for example, a voltage may be applied to the MSS 100 from the state of (D) of FIG. 1 to detect a signal. In this case, it is preferable that the aptamer 17 be immobilized to a carrier which is heavier than the target16.In addition, while the complex is formed in a two-step forming of forming the first complex of the aptamer 15 and the target16 and then forming another complex of the first complex and the aptamer 17 in the description of FIG. 1, as will be described below, the complex may be formed in a one-step forming.

Example embodiments of the MSS in the analysis kit of the present invention will be described. Note here that the present invention is not limited to the following example embodiments. In addition, the descriptions of the respective example embodiments can be referred to each other unless otherwise specified. Furthermore, the configurations of the example embodiments can be combined unless otherwise specified.

### First Example Embodiment

A MSS of the present example embodiment is, as described above, characterized in that it includes an aptamer; a membrane; and a sensor substrate, wherein the aptamer is a nucleic acid molecule that binds to a target and is immobilized to the membrane, the membrane is a membrane that deforms upon binding of the target to the aptamer, the sensor substrate has a support region, the support region supports the membrane and has a piezoresistive element, and the piezoresistive element is an element for detecting deformation of the membrane.

In the MSS of the present example embodiment, the membrane is also referred to as a MSS membrane. As described above, the MSS membrane is not particularly limited as long as it is deformed by the binding of the target to the aptamer and the deformation gives a stress to the piezoresistive element. The membrane is, for example, a thin membrane, and the thickness and the area of each surface thereof are not particularly limited, and are the same as, for example, those of a MSS membrane used in a commercially available MSS. The planar shape of the membrane is, for example, a circle, and specifically, is, for example, a regular circle. The material of the membrane is not particularly limited, and is, for example, a silicon membrane, and a specific example is n-type Si(100).

In the MSS of the present example embodiment, the aptamers are immobilized to the MSS membrane. The aptamers may be immobilized to one surface of the MSS membrane or may be immobilized to both surfaces of the MSS membrane, for example. When the aptamers are immobilized to both surfaces of the MSS membrane, it is preferable that the aptamer on one surface and the aptamer on the other surface be, for example, the same aptamers that bind to the same target. In the following description, the surface of the MSS membrane may be, for example, one surface or both surfaces.

The method of immobilizing the aptamers to the MSS membrane is not particularly limited, and the aptamers may be directly or indirectly immobilized to the MSS membrane. In the former case, for example, by chemically treating the MSS membrane and the aptamers, the aptamers can be immobilized to the MSS membrane by covalent binding or the like. The direct immobilization method may be, for example, a method of utilizing photolithography, and specifically, reference can be made to the specification or the like of U.S. Pat. No. 5,424,186. Another direct immobilization method may be, for example, a method of synthesizing the sensor on the MSS membrane. This method may be, for example, a so-called spot method, and specifically, reference can be made to the specification or the like of U.S. Pat. No. 5,807,522. In the latter case, for example, the aptamer can be immobilized to the MSS membrane via a linker. The type of the linker is not limited in any way, and may be the combination of biotin or a biotin derivative (hereinafter, collectively referred to as biotin) and avidin or an avidin derivative (hereinafter, collectively referred to as avidin). The biotin derivative may be, for example, biocytin and the like, and the avidin derivative may be, for example, streptavidin and the like. The length of the linker may be expressed, for example, by a length of a shortest molecular chain (main chain length) from a functional group on a MSS membrane (e.g., an oxygen atom of a silanol group on a silicon membrane) to an affinity tag such as avidin or an aptamer. The main chain length of the linker is 1 to 20 and is preferably 1 to 15, 1 to 13, 3 to 13, 5 to 13, 1 to 11, 3 to 1 1, 1 to 10, 3 to 10, 1 to 8, 3 to 8, 1 to 5, 1 to 3, 1 or 2 because the sensitivity of the MSS can be improved. Hereinafter, an immobilization method will be exemplified, but the present invention is not limited thereto.

As a first example, the biotin is bonded to one of the MSS membrane and the aptamer, and avidin is bonded to the other. Then, by binding the biotin with the avidin, the aptamer can be indirectly immobilized to the MSS membrane.

Note that, in the first example, the aptamer is indirectly immobilized to the MSS membrane by utilizing the avidin-biotin specific binding, i.e., the affinity of the biotin to the avidin, but the present invention is not limited thereto, and an affinity tag other than the avidin-biotin may be utilized. As the affinity tag, for example, His tag (His×6 tag)-nickel ion, glutathione-S-transferase-glutathione, maltose binding protein-maltose, epitope tag (myc tag, FLAG tag, HA (hemagglutinin) tag)-antibody or antigen-binding fragment can be utilized. Also in the second to fourth examples described below, affinity tags other than the avidin-biotin may be used.

As a second example, the aptamer may be immobilized to the MSS membrane, e.g., via an intervening membrane. The intervening membrane is formed on the MSS membrane, and as in the first example, the biotin is bonded to one of the intervening membrane and the aptamer, and the avidin is bonded to the other, and then the biotin is bonded with the avidin, thereby immobilizing the aptamer to the MSS via the intervening membrane. The intervening membrane is, for example, a membrane of a metal such as gold or the like, and can be formed by depositing the metal with respect to the MSS membrane. The thickness of the intervening membrane is not particularly limited, and is, for example, 10 to 100 nm. The intervening membrane may be made of, for example, one or two or more layers. When forming the intervening membrane with a gold surface, the intervening membrane is preferably made of two layers, for example, and the gold membrane is preferably formed on the MSS membrane via a metal membrane for adhesion (adhesive membrane) from the viewpoint of improving the adhesiveness of the gold membrane. Examples of the metal of the adhesive membrane include titanium and chromium. The thickness of the adhesive membrane is, for example, 0.1 to 10 nm, and the thickness of the gold membrane is, for example, 0.1 to 100 nm. When the biotin is bonded to the intervening membrane, for example, a thiol alkane to which the biotin is bonded may be further used to form a self-assembled monolayer (SAM) of thiol alkane on the surface of the intervening membrane, and the aptamer to which the biotin is bonded may be contacted, and the aptamer may be immobilized by binding the biotin with the avidin.

As a third example, there is a method of binding the streptavidin to the MSS membrane by binding an amino group to the MSS membrane and further binding glutaraldehyde thereto. That is, a silane coupling agent having an amino group is reacted with the MSS membrane to bind an amino group to the MSS membrane. The reaction can be carried out, for example, by applying a solution containing a silane coupling agent having an amino group to the MSS membrane. Further, a crosslinking agent capable of binding an amino group and a main chain or a side chain of an amino acid is reacted with the MSS membrane, or a crosslinking agent such as glutaraldehyde capable of forming a linker between an amino group and a main chain or a side chain of an amino acid is reacted with the MSS membrane, thereby binding one end of a crosslinking agent such as glutaraldehyde to the amino group on the MSS membrane. Specifically, the membrane surface of the MSS membrane after silane coupling is washed, a solution containing a crosslinking agent is applied to the MSS membrane to bind the amino group to the crosslinking agent. The conditions of the crosslinking reaction can be appropriately determined, for example, depending on the type of the crosslinking agent. Next, the avidin is bonded to the other end of the crosslinking agent such as glutaraldehyde. Specifically, the membrane surface of the MSS membrane after crosslinking is washed, and a solution containing the avidin is applied to bind the other end of the crosslinking agent to the main chain or the side chain of the amino acid of the avidin. Then, an aptamer to which the biotin is bonded is brought into contact with the MSS membrane thus treated, and the aptamer can be immobilized by binding the biotin with the avidin.

A silane coupling agent is represented by Y—Si (CH₃)₃, (OR)ₙ, for example. When the silane coupling agent is a silane coupling agent having an amino group, n, R, and Y may be as described below, for example. The “n” is 2 or 3. Examples of the “R” include alkyl groups such as a methyl group, an ethyl group, and the like; and acyl groups such as an acetyl group, a propyl group, and the like. The “Y” is a reactive functional group having an amino group at its end.

Examples of the silane coupling agent having the amino group include N-(2-aminoethyl)-3-aminopropylmethyldimethoxysilane (for example, KBM-602 (produced by Shin-Etsu Silicone Co., Ltd.)), N-(2-aminoethyl)-3-aminopropyltrimethoxysilane (for example, KBM-603 (produced by Shin-Etsu Silicone Co., Ltd.)), 3-aminopropyltriethoxysilane (for example, KBM-903 (produced by Shin-Etsu Silicone Co., Ltd.)), 3-aminopropyltriethoxysilane (for example, KBE-903 (produced by Shin-Etsu Silicone Co., Ltd.)), 3-(2-aminoethylamino)propyltrimethoxysilane (for example, GENIOSIL® GF 91 (produced by Wacker Asahikasei Silicone Co., Ltd.), and 3-(2-aminoethylamino)propylmethyldimethoxysilane (for example, GENIOSIL® GF 95 (produced by Wacker Asahikasei Silicone Co., Ltd.).

The crosslinking agent can be appropriately determined depending on the functional group of the main chain or the side chain of the amino acid to be bonded to the linker. Examples of the functional group include an amino group (—NH₂), a thiol group (—SH), and a carboxyl group (—COOH). The amino group has, for example, an N-terminal of protein or peptide or a side chain of lysine. The thiol group has, for example, a side chain of cysteine. The carboxyl group has, for example, a C-terminal of protein or peptide or a side chain of aspartic acid or glutamic acid.

When an amino group of the main chain or the side chain of the amino acid is utilized, examples of the crosslinking agent include a crosslinking agent having aldehyde groups such as glutaraldehyde and the like at both ends; a crosslinking agent having N-hydroxysuccinimide active esters (N-hydroxysuccinimide reactive groups) such as bis(sulfosuccinimidyl)suberate (BS3), disuscinimidyl glutarate (DSG), disuscinimidyl suberate (DSS), dithiobis(succinimidyl propionate), dithiobis(sulfosuccinimidyl propionate) (DSP), dithiobis(succinimidyl propionate) (DTSP), dithiobis(sulfosuccinimidyl propionate) (DTSSP), dissuccinimidyl tartrate (DST), ethylene glycol bis(succinimidyl succinate) (ESG)), ethylene glycol bis(sulfosuccinimidyl succinate) (Sulfo-ESG), PEGylated bis(sulfosuccinimidyl) (BS(PEG)5, BS(PEG)9, etc.), and the like at both ends; and a crosslinking agent having imide ester reactive groups such as dimethyl adipoimidate (DMA), dimethyl pimelimidate (DMP), dimethyl pimelimidate (DMS), and the like at both ends.

When a thiol group of the side chain of the amino acid is utilized, examples of the crosslinking agent include a crosslinking agent having a N-hydroxysuccinimide active ester and a maleimide group such as N-(6-maleimide caproyloxy)succinimide (EMCS), N-(6-maleimide caproyloxy)sulfosuccinimide (Sulfo-EMCS), N-(8-maleimide capryloxy)succinimide (HMCS), N-(8-maleimide sulfocapryloxy) (Sulflo-HMCS), N-α-maleimidoacet-oxysuccinimide ester (AMAS), N-β-maleimidopropyl-oxysuccinimide ester (BMPS), N-γ-maleimidobutyryl-oxysuccinimide ester (GMBS), N-γ-maleimidobutyryl-oxysulfosuccinimide ester (Sulfo-GMBS), m-maleimidobenzoyl-N-hydroxysuccinimide ester (MBS), m-maleimidobenzoyl-N-hydroxysulfosuccinimide ester (Sulfo-MBS), succinimidyl 4-(N-maleimidomethyl)cyclohexane-1-carboxylate (SMCC), sulfosuccinimidyl 4-(N-maleimidomethyl)cyclohexane-1-carboxylate (Sulfo-SMCC), succinimidyl 4-(p-maleimidophenyl)butyrate (SMPB), sulfosuccinimidyl 4-(N-maleimidophenyl)butyrate (Sulfo-SMPB), Succinimidyl 6-((beta-maleimidopropionamido) hexanoate) (SMPH), succinimidyl 4-(N-maleimidomethyl)cyclohexane-1-carboxy-(6-amidocaproate) (LC-SMCC), N-κ-maleimidoundecanoyl-oxysulfosuccinimide ester (Sulfo-KMUS), or the like at both ends of the molecule; a crosslinking agent having a N-hydroxysuccinimide active ester and a haloacetyl reactive group such as succinimidyl iodoacetate (SIA), succinimidyl 3-(bromoacetamido)propionate (SBAP), succinimidyl(4-iodoacetyl)aminobenzoate (SIAB), sulfosuccinimidyl(4-iodoacetyl)aminobenzoate (Sulfo-SIAB), or the like; and a crosslinking agent having pyridyl dithiol reactive group and a N-hydroxysuccinimide active ester such as succinimidyl 3-(2-pyridyldithio)propionate (SPDP), succinimidyl 6-(3(2-pyridyldithio)propionamido)hexanoate (LC-SPDP), sulfosuccinimidyl 6-(3 (2-pyridyldithio)propionamido)hexanoate (Sulfo-LC- SPDP), 4-succinimidyloxycarbonyl-alpha-methyl-α(2-pyridyldithio)toluene (SMPT), 2-pyridyldithiol-tetraoxatetradecane-N-hydoxysuccinimide (PEG4-SPDP), 2-pyridyldithiol-tetraoxaoctatriacontane-N-hydoxysuccinimide (PEG12-SPDP)), or the like at both ends.

When a carboxyl group of the main chain or the side chain of the amino acid is utilized, examples of the crosslinking agent include dicyclohexylcarbodiimide (DCC), 1-ethyl-3-(3-dimethylaminopropyl)carbodiimide (EDC), N-hydroxysuccinimide (NHS), N-hydroxysulfosuccinimide (Sulfo-NHS), and acetic anhydride. Note that since the amino group and the carboxyl group are directly bonded, DCC, EDC, NHS, Sulfo-NHS, and acetic anhydride do not remain between the carboxyl group and the amino group, and a linker region (group) derived from a crosslinking agent is not formed.

The crosslinking agent is preferably a crosslinking agent in which self-condensation does not substantially occur because the length of the linker can be kept at a substantially constant length or a constant length. “The length of the linker is constant” means that, for example, in a linker of a plurality of aptamers, the length of the linker of each of the aptamers is substantially the same or the same. The length of the linker can be made substantially the same or the same, for example, by making the structure of the linker substantially the same or the same. In the third example, by using such a crosslinking agent, the sensitivity of the MSS can be improved. The improvement of the sensitivity is presumed to be due to the following reasons. Note that the present invention is not limited in any way to the following presumption. By binding of the target to the aptamer, steric hindrance due to the target occurs around the aptamer to which the target is bonded. If the aptamers are immobilized at different distances to the MSS membrane, the target is likely to contact an aptamer present on the distal end side from the MSS membrane. Thus, it is presumed that the target preferentially binds to an aptamer on the distal end side from the MSS membrane. In this case, even if steric hindrance due to the target occurs around the aptamer to which the target is bonded, the other aptamers are less susceptible to steric hindrance due to the target because many of them are present on the MSS membrane side as compared to the aptamer to which the target is bonded. Therefore, even when the target binds to an aptamer, there is a low possibility that the positions of the surrounding aptamers move due to steric hindrance. Thus, when the aptamers are immobilized at different distances to the MSS membrane, there is also a relatively low possibility of distortion of the MSS membrane due to movement of the positions of the surrounding aptamers. On the other hand, when the aptamers are immobilized at substantially the same distance to the MSS membrane, upon binding of the target to the aptamer, the aptamers surrounding the aptamer to which the target is bonded are affected by steric hindrance due to the target. Therefore, the positions of the surrounding aptamers are likely to move, and the distortion of the MSS membrane is relatively likely to occur due to the movement of the positions of the surrounding aptamers. That is, when the aptamers are immobilized at substantially the same distance to the MSS membrane, the binding between one aptamer and the target causes the movement of the positions of the surrounding aptamers on the MSS membrane, thereby increasing the distortion of the MSS membrane. Therefore, when the aptamers are immobilized at substantially the same distance to the MSS membrane, that is, when the length of the linker is kept at a substantially constant length, it is presumed that the sensitivity of the MSS membrane improves.

Specific examples of the crosslinking agent in which the self-condensation does not substantially occur include a crosslinking agent having N-hydroxysuccinimide active esters at both ends, a crosslinking agent having imide ester reaction groups at both ends, a crosslinking agent having a maleimide group and a N-hydroxysuccinimide active ester at both ends of the molecule, a crosslinking agent having a N-hydroxysuccinimide active ester and a haloacetyl reactive group at both ends, a crosslinking agent having a N-hydroxysuccinimide active ester and a pyridyldithiol reactive group at both ends, DCC, EDC, NHS, Sulfo-NHS, and acetic anhydride.

The linker is represented by, for example, the following formula (1). In the following formula (1), M₁ represents an atom bonded to the silane coupling agent on the MSS membrane, L₁ represents a region (group) derived from a silane coupling agent, L₂ represents a region (group) derived from the cross-linking agent, L₂ is optional, and M₂ represents an atom bonded to a cross-linking agent or NH in the affinity tag. Further, NH represents an amine derived from an amino group of a silane coupling agent having an amino group.

M₁-L₁-NH-L₂-M₂ (1)

L₁ is represented by (M₁)-Si(CH₃)₂₋ₘ(OR₄)ₘ-R₁—(NH) or (M₁)-Si(CH₃)₂₋ₘ(OR₄)ₘ—R₂—NH—R₃—(NH), for example. R₁ is a straight or branched alkyl group having 1 to 5 carbon atoms. R₂ and R₃ are, for example, each independently a straight or branched alkyl group having 1 to 5 carbon atoms and may be the same or different from each other. Examples of the alkyl group include a methyl group, an ethyl group, a propyl group, a butyl group, and a pentyl group. R₄ is, for example, a hydrogen atom or a bond, and m is 1 or 2.

When 3-aminopropyltriethoxysilane is used as the silane coupling agent, L₁ is represented by, for example, (M₁)-Si(OR₄)₂—(CH₂)₃—(NH). R₄ is, for example, a hydrogen atom or a bond. Further, when glutaraldehyde is used as the crosslinking agent, L₂ is represented by, for example, (NH)═CH—C₃H₆—CH═(CH(CHO)—C₂H₄—CH)ₙ═CH(CHO)—C₂H₄—C═(M₂).

The length of the linker may be expressed, for example, by a length of a shortest molecular chain (main chain length) from a functional group on a MSS membrane (e.g., an oxygen atom of a silanol group on a silicon membrane) to an affinity tag such as avidin. The main chain length of the linker is 1 to 20 and is preferably 1 to 15, 1 to 13, 3 to 13, 5 to 13, 1 to 11, 3 to 11, 1 to 10, 3 to 10, 1 to 8, 3 to 8, 1 to 5, 1 to 3, 1 or 2 because the sensitivity of the MSS can be improved.

Note that, while the avidin-biotin binding is utilized in the third example, the third example is not limited thereto, and a linker may be directly bonded to a hydroxyl group or a phosphate group of the aptamer. In this case, the aptamer can be immobilized to the MSS membrane by amididizing a phosphate group at the 3′ end to react with the linker.

A fourth example is a method of binding a methacrylic group (—C(═O)—C(CH₃)═CH₂) to the MSS membrane, and binding the streptavidin to the MSS membrane via an amino acid or a derivative of the methacrylic group (hereinafter, referred to as an “amino acid derivative”). That is, a silane coupling agent having a methacrylic group is reacted with the MSS membrane to bind an amino group to the MSS membrane. The reaction can be carried out, for example, by applying a solution containing a silane coupling agent having a methacrylic group to the MSS membrane. Further, an amino acid derivative such as N-acetylcysteine is reacted with the MSS membrane, and then a crosslinking agent capable of forming a linker between a main chain or a side chain of the amino acid derivative and a main chain or a side chain of the amino acid of the avidin is reacted with the MSS membrane thereby binding one end of the crosslinking agent to the amino acid derivative on the MSS membrane. Specifically, the membrane surface of the MSS membrane after treatment with the amino acid derivative is washed, a solution containing a crosslinking agent is applied to the MSS membrane to bind the amino acid derivative to the crosslinking agent. The conditions of the crosslinking reaction can be appropriately determined, for example, depending on the type of the crosslinking agent. Next, the avidin is bonded to the other end of the crosslinking agent. Specifically, the membrane surface of the MSS membrane after crosslinking is washed, and a solution containing avidin is applied to bind the other end of the crosslinking agent to the main chain or the side chain of the amino acid of the avidin. Then, an aptamer to which the biotin is bonded is brought into contact with the MSS membrane thus treated, and the aptamer can be immobilized by binding the biotin with the avidin.

As described above, the silane coupling agent is represented by Y—Si(CH₃)₃₋ₙ(OR)ₙ, for example. When the silane coupling agent is a silane coupling agent having a methacrylic group, n, R, and Y may be as described below, for example. The “n” is 2 or 3. Examples of the “R” include alkyl groups such as a methyl group, an ethyl group, and the like; and acyl groups such as an acetyl group, a propyl group, and the like. The “Y” is a reactive functional group having a methacrylic group at its end.

Examples of the silane coupling agent having a methacrylic group include 3-(methacryloyloxy)propylmethyldimethoxysilane (e.g., KBM-502 (produced by Shin-Etsu Silicone Co., Ltd.)), 3-(methacryloyloxy)propyltrimethoxysilane (e.g., KBM-503 (produced by Shin-Etsu Silicone Co., Ltd.), GENIOSIL® GF31 (produced by Wacker Asahikasei Silicone Co., Ltd..)), 3-(methacryloyloxy)propylmethyldimethoxysilane (e.g., KBE-502 (produced by Shin-Etsu Silicone Co., Ltd.)), and (3-methacryloyloxypropyl)triethoxysilane (e.g., KBE-503 (produced by Shin-Etsu Silicone Co., Ltd.)).

The amino acid or amino acid derivative has, for example, a functional group capable of reacting with a methacrylic group and a carboxyl group. The functional group capable of reacting with the methacrylic group may be, for example, a thiol group (—SH). Examples of the amino acid or amino acid derivative having the thiol group include cysteine; and cysteine having a modified amino group such as N-acetylcysteine.

The crosslinking agent can be appropriately determined depending on, for example, a functional group of the amino acid derivative to be subjected to crosslinking and a functional group of an amino acid of the avidin to be subjected to crosslinking. As a specific example, when two functional groups are amino groups, regarding the crosslinking agent, reference can be made to the description as to a crosslinking agent when utilizing an amino group of a main chain or a side chain of the amino acid in the third example. Also, when one of two functional groups is an amino group and the other is a thiol group, regarding the crosslinking agent, reference can be made to the description as to a crosslinking agent when utilizing a thiol group of a side chain of the amino acid in the third example. Further, when one of two functional groups is an amino group and the other is a carboxyl group, regarding the crosslinking agent, reference can be made to the description as to a crosslinking agent when utilizing a carboxyl group of a main chain or a side chain of the amino acid in the third example.

The crosslinking agent is preferably a crosslinking agent in which self-condensation does not substantially occur because the length of the linker can be kept at a constant length. In the fourth example, by using such a crosslinking agent, the sensitivity of the MSS can be improved by the same mechanism as that described in the third example described above. Specific examples of the crosslinking agent in which self-condensation does not substantially occur include a crosslinking agent having N-hydroxysuccinimide active esters at both ends, a crosslinking agent having imide ester reaction groups at both ends, a crosslinking agent having a maleimide group and a N-hydroxysuccinimide active ester at both ends of the molecule, a crosslinking agent having a N-hydroxysuccinimide active ester and a haloacetyl reactive group at both ends, a crosslinking agent having a N-hydroxysuccinimide active ester and a pyridyldithiol reactive group at both ends, DCC, EDC, NHS, Sulfo-NHS, and acetic anhydride.

The linker is represented by, for example, the following formula (2). In the following formula (2), M₁ represents an atom bonded to the silane coupling agent on the MSS membrane, L₁ represents a region (group) derived from the silane coupling agent, A represents an amino acid derivative, L₂ represents a region (group) derived from the cross-linking agent, L₂ is optional, and M₂ represents an atom bonded to the cross-linking agent or NH in the affinity tag.

M₁-L₁-A-L₂-M₂ (2)

L₁ is represented by, for example, (M₁)—Si(CH₃)₂₋ₘ(OR₄)ₘ—R₅—C(═O)—CH₁(CH₃)₂₋₁(A). R₄ is, for example, a hydrogen atom or a bond. R₅ is a straight or branched alkyl group having 1 to 5 carbon atoms. Examples of the alkyl group include a methyl group, an ethyl group, a propyl group, a butyl group, and a pentyl group. m is 1 or 2. 1 is 0 or 1.

When 3-(methacryloyloxy)propyltrimethoxysilane is used as the silane coupling agent, L₁ is represented by, for example, (M₁)-Si(OR₄)₂—(CH₂)₃—O—C(═O)—C(CH₃)₂-(A). R₄ is, for example, a hydrogen atom or a bond. In addition, when N-acetylcysteine is used as the amino acid derivative, A is represented by, for example, (L₁)—S—CH₂—CH(NH—COCH₃)—C(═O)-(M₂). When acetic anhydride is used as the crosslinking agent, L₂ is not present, for example.

The length of the linker may be expressed, for example, by a length of a shortest molecular chain (main chain length) from a functional group on a MSS membrane (e.g., a silanol group on a silicon membrane) to an affinity tag such as avidin. The main chain length of the linker is 1 to 20 and is preferably 1 to 15, 1 to 13, 1 to 11, 1 to 10, 1 to 8, 1 to 5, 1 to 3, 1 or 2 because the sensitivity of the MSS can be improved.

Note that, while the avidin-biotin binding is utilized in the fourth example, the fourth example is not limited thereto, and a linker may be directly bonded to a hydroxyl group or a phosphate group of the aptamer. In this case, the aptamer can be immobilized to the MSS membrane by amididizing a phosphate group at the 3′ end to react with the linker.

The immobilization site of the aptamer to the MSS membrane is not particularly limited, and may be, for example, the 3′ end or the 5′ end.

In the MSS of the present example embodiment, the sensor substrate has a support region for supporting the MSS membrane, and the support region has a piezoresistive element. The sensor substrate supports the MSS membrane by the support region. In the MSS membrane, for example, the aptamer is immobilized to one or both surfaces facing each other as described above, and supported at the side by the sensor substrate. It is preferable that the sensor substrate partially support the MSS membrane, for example, and in particular, it is preferable that the sensor substrate partially support the side surface of the MSS membrane. In the MSS membrane, the number of positions (supporting portions) supported by the supporting region of the sensor substrate is not particularly limited, and is, for example, four. This description however is a mere example and does not limit the present invention at all.

In the sensor substrate, the support region is, for example, a silicon membrane, and by making any region of the silicon membrane into p-type by doping an impurity, the p-type region (p-type Si) can be functioned as the piezoresistive element. The support region has the piezoresistive element at or in the vicinity of the position supporting the MSS membrane, for example. The sensor substrate may be made entirely of silicon, or only the support region may be a silicon membrane, for example, and materials of the sensor substrate other than the support region having the piezoresistive element are not particularly limited.

In the MSS of the present example embodiment, for example, the sensor substrate has a circuit for applying a voltage. When the support region supports the MSS membrane at a plurality of points and has piezoresistive elements at or in the vicinity of the positions supporting the MSS membrane, for example, the circuit may be, for example, a Wheatstone bridge circuit having a plurality of piezoresistive elements in the support region. As to the MSS of the present example embodiment, for example, by applying a voltage to the Wheatstone bridge circuit, as described above, it is possible to measure the electrical signal accompanying the change in resistance value in the piezoresistive element.

In the MSS of the present example embodiment, for example, the sensor substrate may have a plurality of the support regions, and the plurality of the support regions may each support the MSS membrane. In the MSS of the present example embodiment, the number of the support regions and the number of the MSS membranes to be supported are not particularly limited, and may be one or two or more. When the MSS of the present example embodiment has a plurality of the MSS membranes, the plurality of MSS membranes may be, for example, MSS membranes having aptamers for the same target immobilized thereon, MSS membranes having aptamers for different targets immobilized thereon, or MSS membranes having both the aptamers for the same target and the aptamers for different targets immobilized thereon. Here, the “aptamers for the same target” may be, for example, aptamers having the same sequence for the same target, or aptamers having different sequences for the same target.

When the MSS of the present example embodiment has a plurality of MSS membranes having aptamers for the same target immobilized thereon, for example, a plurality of analyses for the same target can be performed at the same time with one MSS. In addition, when the MSS of the present example embodiment has a plurality of MSS membranes having aptamers for different targets immobilized thereon, for example, analyses for different targets can be performed at the same time with one MSS. In this case, the analysis kit of the present invention has a first binding substance corresponding to each target.

The MSS of the present example embodiment may be in a form in which the MSS membrane is placed on the sensor substrate at the time of use, for example, and the sensor substrate and the MSS membrane may be separately independent before use. In the latter case, for example, the MSS of the present invention may be, for example, a kit including the sensor substrate and the MSS membrane separately and independently.

The MSS of the present example embodiment can measure a change in resistance value due to a stress change of the piezoresistive element in the MSS as an electronic signal by using an existing measurement module in an analysis method described below, for example.

### Second Example Embodiment

A target analysis method of the present embodiment includes the steps of: forming a complex of a target in the sample liquid, the first binding substance, and the second binding substance by bringing a sample liquid into contact with the analysis kit according to the present invention; applying a voltage to the membrane-type surface stress sensor in a liquid phase; and analyzing a target in the sample liquid by measuring a stress change of the piezoresistive element in the membrane-type surface stress sensor. The analysis method of the present embodiment is characterized in that it uses the analysis kit of the present invention, and other steps and conditions and the like are not particularly limited.

The complex-forming can be carried out, for example, by bringing the MSS in the analysis kit into contact with the sample liquid and the first binding substance (by causing the MSS in the analysis kit to coexist with the sample liquid and the first binding substance). In the contacting, for example, contact of the MSS with the sample liquid and the first binding substance may be performed separately or simultaneously.

When the contacting is carried out separately, the complex-forming includes the steps of: forming a first complex of a target in the sample liquid and the second binding substance by bringing the sample liquid into contact with the MSS; and forming a second complex of the first complex and the first binding substance by bringing the membrane-type surface stress sensor after forming the first complex into contact with the first binding substance. The contacting can be carried out by immersing the MSS in the sample liquid. Specifically, the contact in the first complex-forming can be carried out by immersing the support region having the piezoresistive element in the sensor substrate and the MSS membrane supported by the support region in the sample liquid. The conditions for immersing the MSS in the sample liquid are not particularly limited, and for example, the immersion may be carried out for 0.1 to 120 minutes at a temperature of 20 to 35° C. or for 0.1 to 120 minutes at a temperature of 50 to 60° C. When the MSS has a plurality of MSS membranes, for example, a plurality of MSS membranes in the MSS may be simultaneously immersed in the same sample liquid.

Next, in the second complex-forming, the MSS is brought into contact with the first binding substance. The contact with the first binding substance may be carried out, for example, by immersing the support region having the piezoresistive element in the sensor substrate and the MSS membrane supported by the support region in the liquid containing the first binding substance, or by adding the first binding substance to the sample liquid in which the MSS is immersed. The conditions for reacting with the first binding substance are not particularly limited, and reference can be made to the description of the conditions for immersing the MSS.

On the other hand, when the contacting is carried out simultaneously, the sample liquid, the MSS, and the first binding substance are brought into contact with each other to form a complex of a target in the sample liquid, the second binding substance, and the first binding substance. The contacting can be carried out by immersing the MSS into the sample liquid and adding the first binding substance to the sample liquid in parallel. Specifically, the contacting can be carried out by immersing the support region having the piezoresistive element in the sensor substrate and the MSS membrane supported by the support region in the sample liquid and adding the first binding substance to the sample liquid. The conditions for immersing the MSS in the sample liquid are not particularly limited, and for example, the immersion may be carried out for 0.1 to 120 minutes at a temperature of 20 to 35° C. or for 0.1 to 120 minutes at a temperature of 50 to 60° C. When the MSS has a plurality of MSS membranes, for example, a plurality of MSS membranes in the MSS may be simultaneously immersed in the same sample liquid.

In the voltage-applying, as described above, a voltage is applied to the membrane-type surface stress sensor in the liquid phase. The application conditions of the voltage are not particularly limited, and for example, the same conditions as those of a commercially available MSS can be exemplified. The liquid phase may be, for example, a sample liquid in the immersing, or other solvents. In the latter case, the MSS after the immersing may be taken out from the sample liquid and immersed in a new solvent, and then a voltage may be applied. When the MSS is washed for removing a substance that has not bonded to the aptamer in the sample after the immersing in the sample liquid, it is preferable to immerse the MSS in such a new solvent and to apply a voltage as described above. The solvent is not particularly limited, and examples thereof include buffers such as PBS, Tris-HCl, and the like; and water.

The timing of applying a voltage may be at the beginning of the complex-forming, during the complex-forming, or after the complex-forming.

In the analyzing, as described above, the target in the sample liquid is analyzed by measuring the stress change of the piezoresistive element in the MSS. The measurement of the stress change can be performed, for example, by measuring an electrical signal using a commercially available measurement module (for example, MSS-8RM, NANOSENSORS).

### EXAMPLES

### Reference Example 1

Aptamers were immobilized to a commercially available MSS membrane to examine whether the analysis of the target was possible.

(1) Immobilization of Aptamers by Nonspecific Adsorption

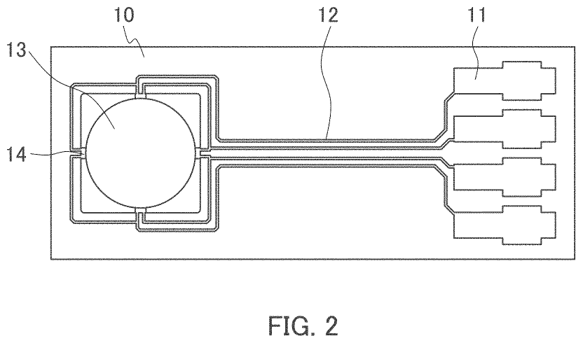

A commercially available MSS (trade name: SD-MSS-1K2G, NANOSENSORS) was used. The configuration of the MSS is shown in the top view of FIG. 2. In the MSS, as shown in FIG. 2, the sensor substrate 10 includes an electrode 11, an aluminum wire 12, a MSS membrane 13, and a piezoresistive element 14, the MSS membrane 13 is connected to the aluminum wire 12 via the piezoresistive element 14, and the aluminum wire 12 is connected to the electrode 11.

An acrylic resin (trade name: Mr. COLOR 62, produced by GSI Creos Corporation) and an epoxy resin (trade name: PM 165-R Hi, produced by CEMEDINE CO., LTD.) were applied to the aluminum wire on the sensor substrate to perform a waterproofing treatment. The sensor substrate after the treatment was connected to a substrate with a connector (trade name: IFB-FFC (0.5) 4P-B, produced by AITENDO) so that the electrode was inserted into the sensor substrate. Further, the waterproofing treatment was applied to the entire connector of the substrate with the connector by applying the same resin as described above to the exposed portion of the metal, the gap connected to the metal portion, and the like to fill up with the resin.

Next, 1 μl of a streptavidin solution was added dropwise to the back surface (the surface opposite to the surface on which the aluminum wire was formed) of the MSS membrane of the sensor substrate, and the sensor substrate was allowed to stand for 1 hour at room temperature under a water vapor atmosphere (100% (relative humidity)). The streptavidin solution was prepared by suspending in 1×PBS (pH 7.4) so as to achieve a streptavidin concentration of 2%. Next, after washing the back surface with the PBS, 3 μl of the streptavidin solution was added dropwise to the surface of the MSS membrane, allowed to stand under the same conditions, and further washed with the PBS. Then, the sensor substrate was dried at room temperature for 10 minutes.

Two of the sensor substrates were treated in this manner, and aptamers were further bonded to one of the sensor substrates to provide the MSS of Reference Example 1A, and poly-T was further added to the other of the sensor substrates to provide the MSS of Negative Control (NC) 1A.

In other words, 3 μl of an aptamer solution was added dropwise to the surface of the one of the sensor substrates and allowed to stand for 1 hour at room temperature under a water vapor atmosphere (100% (relative humidity)). The aptamer solution was prepared by suspending thrombin aptamer having a biotin tag added to the 3′ end (SEQ ID NO: 1: GGTTGGTGTGGTTGGTTTTT-biotin-3′) in the PBS so as to have a final concentration of 1 μ/mol. Since the thrombin aptamer has a biotin tag, if streptavidin is bonded to the surface of the MSS membrane, the thrombin aptamer is immobilized to the surface of the MSS membrane by the binding between the biotin and the streptavidin. This was referred to as the MSS of Reference Example 1A.

3 μl of a poly T solution was added dropwise to the surface of the other of the sensor substrates and allowed to stand for 1 hour at room temperature under a water vapor atmosphere (100% (relative humidity)). The poly T solution was prepared by suspending DNA of poly T having a biotin tag added to the 3′ end (SEQ ID NO: 2: TTTTTTTTTTTTTTTTTTTT-biotin-3′) in the PBS so as to have a final concentration of 1 μmol/l. Since the poly T has a biotin tag, if streptavidin is bonded to the surface of the MSS membrane, the poly T is immobilized to the surface of the MSS membrane by the binding between the biotin and the streptavidin. This was referred to as the MSS of NC1A.

2) Immobilization of Aptamers by Gold Deposition

The same treatment as in (1) above was performed, unless otherwise indicated. That is, the waterproofing treatment was applied to the aluminum wire on the sensor substrate of the commercially available MSS, then the electrode of the sensor substrate was masked, titanium deposition was performed on the entire surface of the sensor substrate including the MSS membrane, and then gold deposition was further performed. A titanium thin membrane having a thickness of about 5 nm was formed by the titanium deposition, and a gold thin membrane having a thickness of about 100 nm was formed by the gold deposition. The sensor substrate was then washed with ethanol.

Next, the entire sensor substrate was immersed in 100 μmol/l BiotinSAM ethanol solution (Dojindo Laboratories), allowed to stand at room temperature for 1 hour, and further washed with ethanol. Then, in the same manner as in (1) above, the sensor substrate was connected to the substrate with a connector, and the entire connector was subjected to the waterproofing treatment.

Next, 1 μl of a 0.5% streptavidin solution was added dropwise to one surface (the surface on which the aluminum wire was formed) of the MSS membrane of the sensor substrate, and the sensor substrate was allowed to stand for 0.5 hours at room temperature under a water vapor atmosphere (100% (relative humidity)). Next, the sensor substrate was washed with the PBS, and then was dried at room temperature for 10 minutes.

Two of the sensor substrates were treated in this manner, and aptamers were further bonded to one of the sensor substrates to provide the MSS of Reference Example 1B, and poly-T was further added to the other of the sensor substrates to provide the MSS of NC1B.

In other words, 3 μl of an aptamer solution was added dropwise to the surface of the one of the sensor substrates and allowed to stand for 1 hour at room temperature under a water vapor atmosphere (100% (relative humidity)). The aptamer solution was prepared by suspending the thrombin aptamer in the PBS so as to have a final concentration of 5 μmold After standing, the sensor substrate was further immersed in PBS containing 1% BSA, allowed to stand at room temperature for 50 minutes, and washed with the PBS. Since the thrombin aptamer has a biotin tag, if streptavidin is bonded to the surface of the MSS membrane, the thrombin aptamer is immobilized to the surface of the MSS membrane by the binding between the biotin and the streptavidin. This was referred to as the MSS of Reference Example 1B.

3 μl of a poly T solution was added dropwise to the surface of the other of the sensor substrates and allowed to stand for 1 hour at room temperature under a water vapor atmosphere (100% (relative humidity)). The poly T solution was prepared by suspending DNA of the poly T in the PBS so as to have a final concentration of 5 μmol/l. After standing, the sensor substrate was further immersed in PBS containing 1% BSA, allowed to stand at room temperature for 50 minutes, and washed with the PBS. Since the poly T has a biotin tag, if streptavidin is bonded to the surface of the MSS membrane, the poly T is immobilized to the surface of the MSS membrane by the binding between the biotin and the streptavidin. This was referred to as the MSS of NC1B.

(3) Immobilization of Aptamers by Silane Coupling-1

The sensor substrate of the commercially available MSS was washed with ethanol, the end of the sensor substrate on which the MSS membrane was placed was immersed in a silane coupling solution and allowed to stand for 20 minutes at room temperature. The composition of the silane coupling agent was as follows: 8 ml of ethanol, 200 μl of acetic acid, 10 μl of APTMS (3-aminopropyltriethoxysilane), and 1.8 ml of pure water. Then, the immersed end of the sensor substrate was washed with pure water, and the treatment was performed at 110° C. for 1.5 hours.

In the same manner as in the (1), the waterproofing treatment was applied to the aluminum wire on the sensor substrate, then the sensor substrate was connected to the substrate with a connector, and the entire connector was subjected to the waterproofing treatment.

Next, 1 μl of a 14% glutaraldehyde solution was added dropwise to one surface (the surface on which the aluminum wire was formed) of the MSS membrane of the sensor substrate, and the sensor substrate was allowed to stand for 0.75 hours at room temperature under a water vapor atmosphere (100% (relative humidity)). Next, the sensor substrate was washed with the PBS, and then was dried at room temperature for 10 minutes. Further, 1 μl of a 0.5% streptavidin solution was added dropwise to the same surface of the MSS membrane of the sensor substrate, and the sensor substrate was allowed to stand for 0.5 hours at room temperature under a water vapor atmosphere (100% (relative humidity)). Next, the surface of the end of the sensor substrate on which the MSS membrane was placed was washed with 0.1 mol/l Tris-HCl (pH8), and then further immersed in 0.1 mol/l Tris-HCl (pH8) and allowed to stand at room temperature for 15 minutes. Thereafter, the end of the sensor substrate was washed with the PBS.

Two of the sensor substrates were treated in this manner, and aptamers were further bonded to one of the sensor substrates to provide the MSS of Reference Example 1C, and poly-T was further added to the other of the sensor substrates to provide the MSS of NC1C.

In other words, 3 μl of an aptamer solution was added dropwise to the surface of the one of the sensor substrates and allowed to stand for 1 hour at room temperature under a water vapor atmosphere (100% (relative humidity)). The aptamer solution was prepared by suspending the thrombin aptamer in the PBS so as to have a final concentration of 5 μmol/l. After standing, the sensor substrate was further immersed in PBS containing 1% BSA, allowed to stand at room temperature for 30 minutes, and washed with the PBS. Since the thrombin aptamer has a biotin tag, if streptavidin is bonded to the surface of the MSS membrane, the thrombin aptamer is immobilized to the surface of the MSS membrane by the binding between the biotin and the streptavidin. This was referred to as the MSS of Reference Example 1C.

3 μl of a poly T solution was added dropwise to the surface of the other of the sensor substrates and allowed to stand for 1 hour at room temperature under a water vapor atmosphere (100% (relative humidity)). The poly T solution was prepared by suspending DNA of the poly T in the PBS so as to have a final concentration of 5 μmol/l. After standing, the sensor substrate was further immersed in PBS containing 1% BSA, allowed to stand at room temperature for 30 minutes, and washed with the PBS. Since the poly T has a biotin tag, if streptavidin is bonded to the surface of the MSS membrane, the poly T is immobilized to the surface of the MSS membrane by the binding between the biotin and the streptavidin. This was referred to as the MSS of NC1C.

(4) Immobilization of Aptamers by Silane Coupling-2

The sensor substrate of the commercially available MSS of Example 1 (1) was washed with ethanol, the end of the sensor substrate on which the MSS membrane was placed was rinsed with about 100 μl of a silane coupling solution and left at room temperature for 1.5 hours. The composition of the silane coupling agent was as follows: 8 ml of ethanol, 200 μl of acetic acid, 100 μl of APTMS (trimethoxylyl 3-propylmethacrylic acid (3-(methacryloyloxy)propyltrimethoxysilane), and 1.8 ml of pure water. Then, the sensor substrate was washed with ethanol and dried at room temperature for 5 minutes.

Next, 1 μl of a N-acetylcysteine solution was added dropwise to one surface (the surface on which the aluminum wire was formed) of the MSS membrane of the sensor substrate, and irradiated with UV (NS365L-6SMG, manufactured by Nitride Semiconductors Co,. Ltd.) for several minutes. Under a steam atmosphere (100% (relative humidity)), irradiation was performed at room temperature until dry. Thereafter, the sensor substrate was washed with pure water and dried at room temperature.

Next, the sensor portion of the sensor substrate was immersed in an acetic anhydride solution (10% acetic anhydride, 90% acetonitrile) and reacted at 60° C. for 0.5 hours. After the reaction, the sensor substrate was washed with acetonitrile.

Next, the sensor substrate was dried at room temperature for 10 minutes. Further, 1 μl of a 0.5% streptavidin solution was added dropwise to the same surface of the MSS membrane of the sensor substrate, and the sensor substrate was allowed to stand for 1.5 hours at room temperature under a water vapor atmosphere (100% (relative humidity)). After standing, the sensor substrate was washed with the PBS.

In the same manner as in Example 1 (1), the waterproofing treatment was applied to the aluminum wire on the sensor substrate, then the sensor substrate was connected to the substrate with a connector, and the entire connector was subjected to the waterproofing treatment.

Two of the sensor substrates were treated in this manner, and aptamers were further bonded to one of the sensor substrates to provide the MSS of Reference Example 1D, and poly-T was further added to the other of the sensor substrates to provide the MSS of NC1D.

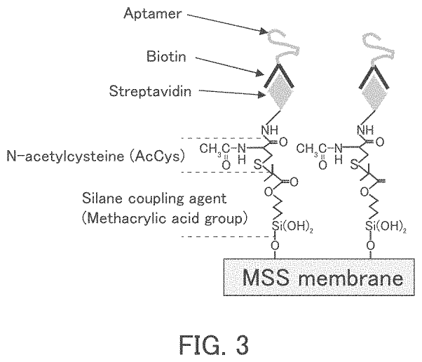

In other words, 3 μl of an aptamer solution was added dropwise to the surface of the one of the sensor substrates and allowed to stand for 1 hour at room temperature under a water vapor atmosphere (100% (relative humidity)). The aptamer solution was prepared by suspending the thrombin aptamer in the PBS so as to have a final concentration of 5 μmol/l. After standing, the sensor substrate was further immersed in PBS containing 1% BSA, allowed to stand at room temperature for 30 minutes, and washed with the PBS. Since the thrombin aptamer has a biotin tag, if streptavidin is bonded to the surface of the MSS membrane, as shown in FIG. 3, the thrombin aptamer is immobilized to the surface of the MSS membrane by the binding between the biotin and the streptavidin. This was referred to as the MSS of Reference Example 1D. Note that the MSS of Reference Example 1D corresponds to the MSS having aptamers immobilized thereon at substantially the same distances with respect to the MSS membrane.

3 μl of a poly T solution was added dropwise to the surface of the other of the sensor substrates and allowed to stand for 1 hour at room temperature under a water vapor atmosphere (100% (relative humidity)). The poly T solution was prepared by suspending DNA of the poly T in the PBS so as to have a final concentration of 5 μmol/l. After standing, the sensor substrate was further immersed in PBS containing 1% BSA, allowed to stand at room temperature for 30 minutes, and washed with the PBS. Since the poly T has a biotin tag, if streptavidin is bonded to the surface of the MSS membrane, the poly T is immobilized to the surface of the MSS membrane by the binding between the biotin and the streptavidin. This was referred to as the MSS of NC1D.

(5) Detection of Electrical Signal

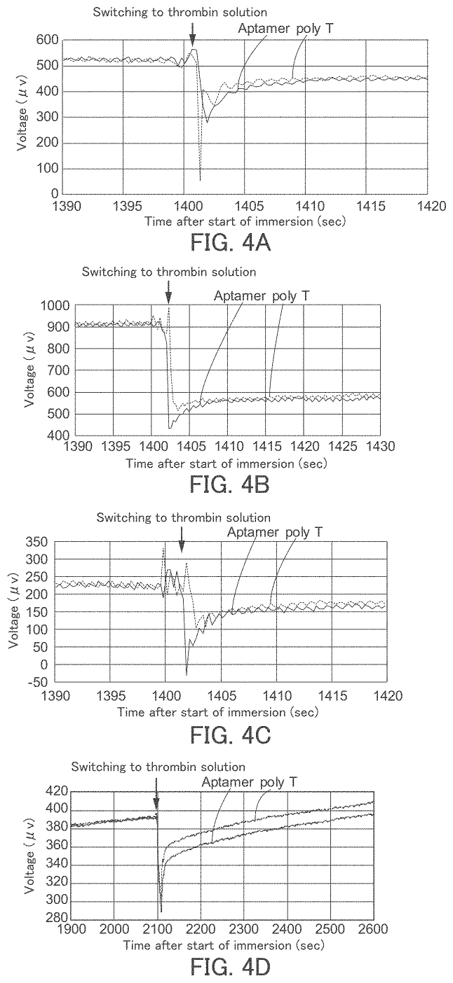

Each set of the MSSs of Reference Examples 1A to 1D, and the MSSs of NC1A to NC1D was immersed in a sample liquid at the same time, and a voltage was applied to measure a voltage change accompanying a stress change. Specifically, first, the end of the MSS including the MSS membrane was immersed in the PBS, a voltage was applied to the MSS, and the MSS was left to stand until the signal of the voltage was stabilized. Then, at a measurement time of 1400 seconds or 2100 seconds when the signal of the voltage was sufficiently stabilized, the solution of the immersion of the MSS was switched from the sample liquid to the thrombin solution, and the signal of the voltage was continuously measured. The thrombin solution was prepared by mixing a thrombin reagent (αThrombin, Human, manufactured by Funakoshi Co., Ltd.) in the PBS so as to have a final concentration of 240 nmol/l. The results are shown in FIG. 4.

FIG. 4 shows graphs showing the voltage. In FIG. 4, (A) shows the results of Reference Example 1A and NC1A, (B) shows the results of Reference Example 1B and NC1B, (C) shows the results of Reference Example 1C and NC1C, and (D) shows the results of Reference Example 1D and NC1D. In FIG. 4, the horizontal axis indicates the elapsed time after switching to the thrombin solution, and the vertical axis indicates the voltage (μV). As shown in (A) to (D) of FIG. 4, in all MSSs, the MSS of Reference Example showed a lower voltage on average compared to the MSS of Control. Further, in a state of being stabilized after the voltage drop, when comparing the difference between the voltage of the MSS of Reference Example and the voltage of the MSS of Control, the average value of the difference in voltage was as follows: Reference Example 1D (about 15 μV)>Reference Example 1A (about 13 μV)>Reference Example 1C (about 10 μV)≥Reference Example 1B (about 7 μV).

From the above, it was found that the target can be detected by arranging the second binding substance on the MSS membrane.

### Example 1

It was examined that the signal was enhanced by the analysis method using the analysis kit of the present invention.

(1) Preparation of MSS

A MSS for use in the analytical kit of Example (MSS of Example 1) was prepared in the same manner as in Reference Example 1A. Further, the MSS of Control (MSS of Comparative Example 1) was prepared in the same manner as in NC1A.

(2) Preparation of Immobilized Aptamers

As a carrier, avidin-labeled magnetic beads (particle size: 2.8 μm, Dynabeads M-280 Streptavidin, manufactured by VERITAS Corporation.) were used. Next, a thrombin aptamer having a 5′ end labeled with biotin (SEQ ID NO: 3: 5′-biotin-AGTCCGTGGTAGGGCAGGTTGGGGTGACT-3′) was suspended in the PBS so as to have a final concentration of 1 mol/l. Since the thrombin aptamer has a biotin tag, an aptamer immobilized to a carrier can be prepared by binding streptavidin of the magnetic bead. It is to be noted that the thrombin aptamer of SEQ ID NO: 3 was confirmed to bind to different thrombin position from the thrombin aptamer of SEQ ID NO: 1 and not compete with the thrombin aptamer of SEQ ID NO: 1. Then, 100 μl of avidin-labeled magnetic beads (Dynabeads M-280 Streptavidin) and 2 μl of an aptamer solution (100 μmol/l) were mixed and allowed to stand at room temperature for 0.5 hours. Thus, an aptamer immobilized to the carrier was prepared.

(3) Detection of Electrical Signal

A set of the MSS of Example 1 and the MSS of Comparative Example 1 was immersed in a sample liquid simultaneously, and a voltage was applied to measure a voltage change accompanying a stress change. Specifically, first, the end of the MSS including the MSS membrane was immersed in the PBS, and a voltage was applied to the MSS, and the MSS was left to stand until the signal of the voltage was stabilized. Then, at a measurement time of 1400 seconds when the signal of the voltage was sufficiently stabilized, the solution of the immersion of the MSS was switched from the sample liquid to the thrombin solution, and the signal of the voltage was continuously measured. The thrombin solution was prepared by mixing a thrombin reagent (aThrombin, Human, manufactured by Funakoshi Co., Ltd.) in the PBS so as to have a final concentration of 240 nmol/l.

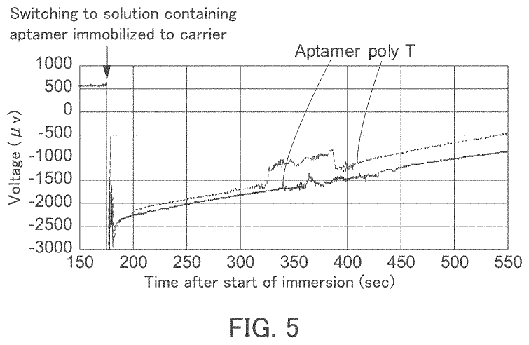

After the voltage signal was sufficiently stabilized in the sample liquid, the sample liquid was switched to a solution containing an aptamer immobilized to the carrier, and the voltage signal was subsequently measured. The results are shown in FIG. 5.

FIG. 5 is a graph showing the measurement of the voltage over time. In FIG. 5, the horizontal axis indicates the elapsed time after switching to the thrombin solution, and the vertical axis indicates the voltage (N). It should be noted that the voltage fluctuation in the MSS of Comparative Example 1 around 350 seconds in FIG. 5 occurs because the measurer erroneously touched the MSS of Comparative Example 1. As shown in FIG. 5, the analysis using the MSS (Aptamer) of Example 1 resulted in about 70 to 80 differences in voltage values as compared with the analysis using the MSS (poly T) of Comparative Example 1, and it was found that the analysis kit and analysis method of the present invention can analyze targets. In Reference Example 1, the MSSs of Reference Examples 1A to 1D have differences of about 5 to 10 times in voltage value as compared with the MSSs of NC1A to 1D. Thus, it was found that, according to the analysis kit and the analysis method of the present invention, a strong electrical signal of about 5 times or more can be obtained as compared with a MSS having a binding substance capable of binding to a target immobilized thereon.

From the above, it was verified that the signal was enhanced by the analysis method using the analysis kit of the present invention as compared with the analysis using a MSS having a binding substance capable of binding to a target immobilized thereon.

While the present invention has been described above with reference to illustrative example embodiments and examples, the present invention is by no means limited thereto. Various changes and variations that may become apparent to those skilled in the art may be made in the configuration and specifics of the present invention without departing from the scope of the present invention.

This application claims priority from Japanese Patent Application Nos. 2020-056896 filed on Mar. 26, 2020. The entire subject matter of the Japanese Patent Applications is incorporated herein by reference.

(Supplementary Notes)

Some or all of the above example embodiments and examples may be described as in the following Supplementary Notes, but are not limited thereto.

### (Supplementary Note 1)

A target analysis kit, including:

a first binding substance that binds to a target; and

a membrane-type surface stress sensor, wherein

the membrane-type surface stress sensor includes:
a second binding substance;
a membrane; and
a sensor substrate, wherein
the second binding substance is a substance that binds to a target and is immobilized to the membrane,
the membrane is a membrane that deforms upon binding of the target to the second binding substance,
the sensor substrate has a support region,
the support region supports the membrane and has a piezoresistive element, and
the piezoresistive element is an element for detecting deformation of the membrane.

### (Supplementary Note 2)

The analysis kit according to Supplementary Note 1, wherein

the first binding substance is an aptamer or an antibody.

### (Supplementary Note 3)

The analysis kit according to Supplementary Note 1 or 2, wherein

the first binding substance is immobilized to a carrier.

### (Supplementary Note 4)

The analysis kit according to Supplementary Note 3, wherein

the carrier is a bead.

### (Supplementary Note 5)

The analysis kit according to any one of Supplementary Notes 1 to 4, wherein

the second binding substance is an aptamer or an antibody.

### (Supplementary Note 6)

The analysis kit according to any one of Supplementary Notes 1 to 5, wherein

the membrane is a silicon membrane.

### (Supplementary Note 7)

The analysis kit according to any one of Supplementary Notes 1 to 6, wherein

the support region partially supports the membrane.

### (Supplementary Note 8)

The analysis kit according to any one of Supplementary Notes 1 to 7, wherein

the second binding substances are immobilized to one surface of the membrane.

### (Supplementary Note 9)

The analysis kit according to any one of Supplementary Notes 1 to 7, wherein

the second binding substances are immobilized to both surfaces of the membrane.

### (Supplementary Note 10)

The analysis kit according to any one of Supplementary Notes 1 to 9, wherein

the second binding substances are immobilized to the membrane via a conjugate of avidin or an avidin derivative and biotin or a biotin derivative.

### (Supplementary Note 11)

The analysis kit according to any one of Supplementary Notes 1 to 9, including:

a metal membrane on the surface of the membrane, wherein

the second binding substances are immobilized to the surface of the membrane via the metal membrane.

### (Supplementary Note 12)

The analysis kit according to any one of Supplementary Notes 1 to 10, wherein

the second binding substances are immobilized to the surface of the membrane via a linker.

### (Supplementary Note 13)

The analysis kit according to Supplementary Note 12, wherein

a length of the linker is substantially constant.

### (Supplementary Note 14)

The analysis kit according to Supplementary Note 12 or 13, wherein

the linker contains a silane coupling agent.

### (Supplementary Note 15)

The analysis kit according to any one of Supplementary Notes 1 to 14, wherein

the sensor substrate has a plurality of support regions, and

the plurality of support regions each support the membrane.

### (Supplementary Note 16)

The analysis kit according to Supplementary Note 15, wherein

the plurality of membrane-type surface stress sensors includes sensors having second binding substances for different targets immobilized thereon, and

the analysis kit includes first binding substances for different targets as the first binding substance.

### (Supplementary Note 17)

The analysis kit according to any one of Supplementary Notes 1 to 16, wherein

the sensor substrate has a circuit,

the support region has a plurality of piezoresistive elements, and

the circuit is a Wheatstone bridge circuit having the plurality of piezoresistive elements.

### (Supplementary Note 18)

A method for analyzing a target, including the steps of:

forming a complex of a target in the sample liquid, the first binding substance, and the second binding substance by bringing a sample liquid into contact with the analysis kit according to any one of Supplementary Notes 1 to 17;

applying a voltage to the membrane-type surface stress sensor in a liquid phase; and

analyzing the target in the sample liquid by measuring a stress change of the piezoresistive element in the membrane-type surface stress sensor.

### (Supplementary Note 19)

The analysis method according to Supplementary Note 18, wherein

the complex-forming includes the steps of:

forming a first complex of a target in the sample liquid and the second binding substance by bringing the sample liquid into contact with the membrane-type surface stress sensor; and

forming a second complex of the first complex and the second binding substance by bringing the membrane-type surface stress sensor after forming the first complex into contact with the second binding substance.

### (Supplementary Note 20)

The analysis method according to Supplementary Note 18 or 19, wherein

in the voltage-applying, the liquid phase is the sample liquid, and

the voltage-applying is performed in parallel with the complex-forming.

### INDUSTRIAL APPLICABILITY

According to the present invention, a strong electrical signal can be obtained as compared with a MSS having a binding substance capable of binding to a target immobilized thereon. Therefore, the present invention is useful, for example, in an analysis field of a sample or the like, a medical field, and the like.

### REFERENCE SIGNS LIST

10: sensor substrate

11: electrode

12: aluminum wire

13: MSS membrane

14: piezoresistive element

100: MSS

What is claimed is:

1. A target analysis kit, comprising:
a first binding substance that binds to a target; and
a membrane-type surface stress sensor, wherein
the membrane-type surface stress sensor comprises:
a second binding substance;
a membrane; and
a sensor substrate, wherein
the second binding substance is a substance that binds to a target and is immobilized to the membrane,
the membrane is a membrane that deforms upon binding of the target to the second binding substance,
the sensor substrate has a support region,
the support region supports the membrane and has a piezoresistive element, and
the piezoresistive element is an element for detecting deformation of the membrane.

2. The analysis kit according to claim 1, wherein
the first binding substance is an aptamer or an antibody.

3. The analysis kit according to claim 1, wherein the first binding substance is immobilized to a carrier.

4. The analysis kit according to claim 3, wherein
the carrier is a bead.

5. The analysis kit according to claim 1, wherein the second binding substance is an aptamer or an antibody.

6. The analysis kit according to claim 1, wherein the membrane is a silicon membrane.

7. The analysis kit according to claim 1, wherein the support region partially supports the membrane.

8. The analysis kit according to claim 1, wherein the second binding substances are immobilized to one surface of the membrane.

9. The analysis kit according to claim 1, wherein
the second binding substances are immobilized to both surfaces of the membrane.

10. The analysis kit according to claim 1, wherein
the second binding substances are immobilized to the membrane via a conjugate of avidin or an avidin derivative and biotin or a biotin derivative.

11. The analysis kit according to claim 1, comprising:
a metal membrane on the surface of the membrane, wherein
the second binding substances are immobilized to the surface of the membrane via the metal membrane.

12. The analysis kit according to claim 1, wherein
the second binding substances are immobilized to the surface of the membrane via a linker.

13. The analysis kit according to claim 12, wherein
a length of the linker is substantially constant.

14. The analysis kit according to claim 12, wherein
the linker contains a silane coupling agent.

15. The analysis kit according to claim 1, wherein the sensor substrate has a plurality of support regions, and the plurality of support regions each support the membrane.

16. The analysis kit according to claim 15, wherein
the plurality of membrane-type surface stress sensors includes sensors having second binding substances for different targets immobilized thereon, and
the analysis kit comprises first binding substances for different targets as the first binding substance.

17. The analysis kit according to claim 1, wherein
the sensor substrate has a circuit,
the support region has a plurality of piezoresistive elements, and
the circuit is a Wheatstone bridge circuit having the plurality of piezoresistive elements.

18. A method for analyzing a target, comprising:
forming a complex of a target in the sample liquid, the first binding substance, and the second binding substance by bringing a sample liquid into contact with the analysis kit according to claim 1;
applying a voltage to the membrane-type surface stress sensor in a liquid phase; and
analyzing the target in the sample liquid by measuring a stress change of the piezoresistive element in the membrane-type surface stress sensor.

19. The analysis method according to claim 18, wherein
the complex-forming comprising:
forming a first complex of a target in the sample liquid and the second binding substance by bringing the sample liquid into contact with the membrane-type surface stress sensor; and
forming a second complex of the first complex and the first binding substance by bringing the membrane-type surface stress sensor after forming the first complex into contact with the first binding substance.

20. The analysis method according to claim 18, wherein in the voltage-applying, the liquid phase is the sample liquid, and the voltage-applying is performed in parallel with the complex-forming.


SEQUENCE LISTING (3 sequences)

SEQ ID NO: 1 - DNA, 20 bp, Artificial Sequence
  Thrombin aptamer
ggttggtgtggttggttttt

SEQ ID NO: 2 - DNA, 20 bp, Artificial Sequence
  poly T
tttttttttttttttttttt

SEQ ID NO: 3 - DNA, 29 bp, Artificial Sequence
  thrombin aptamer
agtccgtggtagggcaggttggggtgact